(19) United States
(12) Patent Application Publication
(10) Pub. No.: US 2012/0003727 A1
(43) Pub. Date: Jan. 5, 2012

(54) Immunoassay Device for Detecting Antibodies and Antigens

(76) Inventor: Javanbakhsh Esfandiari, Stony Brook, NY (US)

(21) Appl. No.: 12/965,258

(22) Filed: Dec. 10, 2010

Related U.S. Application Data
(63) Continuation-in-part of application No. 11/908,071, filed on Sep. 7, 2007, now Pat. No. 7,879,597.
(60) Provisional application No. 61/338,303, filed on Feb. 16, 2010.

(51) Int. Cl. C12M 1/34

(52) U.S. Cl. 435/287.2

(57) ABSTRACT

A fourth generation immunoassay device includes first, second, third, and fourth sorbent or bibulous materials defining first, second, third and fourth horizontal flow paths. The first and second flow paths are for migration of first and second conjugates while the third and fourth flow paths are for the migration of a liquid sample. A first test area for detecting the presence of one or more different antibodies is located at the juncture of the first and third flow paths, and a second test area for detecting the presence of one or more different antigens is located at the juncture of the second and fourth flow paths. A housing is optionally provided for the sorbent materials with an opening for receiving a sample, one or more openings for receiving buffer solution or a conjugate-buffer subcomplex. The housing may also have viewing windows above the detection areas.

### PRIORITY AND RELATED APPLICATIONS

This application claims benefit from provisional application 61/338,303 filed Feb. 16, 2009. This application is a continuation-in-part of U.S. Ser. No. 11/908,071 filed Sep. 7, 2007 entitled “Dual Path Immunoassay Device” which is the national stage application of PCT/US2006/008688 filed Mar. 10, 2006 published as WO 2006/099191 A2.

### BACKGROUND OF THE INVENTION

1. Field of the Invention

This invention relates broadly to immunoassay devices and the methods for their use. More particularly, this invention relates to chromatographic rapid test strips for detection of a ligand in a body fluid. Even more particularly, this invention relates to “fourth generation assays” which allow the simultaneous detection of an antigen and an antibody for clinical diagnostic purposes.

2. State of the Art

Many types of ligand-receptor assays have been used to detect the presence of various substances, often generally called ligands, in body fluids such as blood, urine, or saliva. These assays involve antigen antibody reactions, synthetic conjugates comprising radioactive, enzymatic, fluorescent, or visually observable polystyrene or metal sol tags, and specially designed reactor chambers. In all these assays, there is a receptor, e.g., an antibody, which is specific for the selected ligand or antigen, and a means for detecting the presence, and in some cases the amount, of the ligand-receptor reaction product. Some tests are designed to make a quantitative determination, but in many circumstances all that is required is a positive/negative qualitative indication. Examples of such qualitative assays include blood typing, most types of urinalysis, pregnancy tests, and AIDS tests. For these tests, a visually observable indicator such as the presence of agglutination or a color change is preferred.

Even the qualitative assays must be very sensitive because of the often small concentration of the ligand of interest in the test fluid. False positives can also be troublesome, particularly with agglutination and other rapid detection methods such as dipstick and color change tests. Because of these problems, so-called “sandwich” assays and other sensitive detection mechanisms which use metal sols or other types of colored particles have been developed.

In a “sandwich” assay, a target analyte such as an antigen is “sandwiched” between a labeled antibody and an antibody immobilized onto a solid support. The assay is read by observing the presence and/or amount of bound antigen-labeled antibody complex. In a “competition” immunoassay, antibody bound to a solid surface is contacted with a sample containing an unknown quantity of antigen analyte and with labeled antigen of the same type. The amount of labeled antigen bound on the solid surface is then determined to provide an indirect measure of the amount of antigen analyte in the sample.

Because these and other assays can detect both antibodies and antigens, they are generally referred to as immunochemical ligand-receptor assays or simply immunoassays.

Solid phase immunoassay devices, whether of the sandwich or competition type, provide sensitive detection of an analyte in a biological fluid sample such as blood, urine, or saliva. Solid phase immunoassay devices incorporate a solid support to which one member of a ligand-receptor pair, usually an antibody, antigen, or hapten, is bound. Common early forms of solid supports were plates, tubes, or beads of polystyrene which were well known from the fields of radioimmunoassay and enzyme immunoassay. In the last decade, a number of porous materials such as nylon, nitrocellulose, cellulose acetate, glass fibers, and other porous polymers have been employed as solid supports.

A number of self-contained immunoassay kits using porous materials as solid phase carriers of immunochemical components such as antigens, haptens, or antibodies have been described. These kits are usually dipstick, flow-through, or migratory in design.

In the more common forms of dipstick assays, as typified by home pregnancy and ovulation detection kits, immunochemical components such as antibodies are bound to a solid phase. The assay device is “dipped” for incubation into a sample suspected of containing unknown antigen analyte. Enzyme-labeled antibody is then added, either simultaneously or after an incubation period. The device is then washed and inserted into a second solution containing a substrate for the enzyme. The enzyme-label, if present, interacts with the substrate, causing the formation of colored products which either deposit as a precipitate onto the solid phase or produce a visible color change in the substrate solution.

Flow-through type immunoassay devices were designed to obviate the need for extensive incubation and cumbersome washing steps associated with dipstick assays. Valkirs et al., U.S. Pat. No. 4,632,901, disclose a device comprising antibody (specific to a target antigen analyte) bound to a porous membrane or filter to which is added a liquid sample. As the liquid flows through the membrane, target analyte binds to the antibody. The addition of sample is followed by addition of labeled antibody. The visual detection of labeled antibody provides an indication of the presence of target antigen analyte in the sample.

Korom et al., EP-A 0 299 359, discloses a variation in the flow-through device in which the labeled antibody is incorporated into a membrane which acts as a reagent delivery system.

The requirement of multiple addition and washing steps with dipstick and flow-through type immunoassay devices increases the likelihood that minimally trained personnel and home users will obtain erroneous assay results.

In migration type assays, a membrane is impregnated with the reagents needed to perform the assay. An analyte detection zone is provided in which labeled analyte is bound and assay indicia is read. See, for example, Tom et al., U.S. Pat. No. 4,366,241, and Zuk, et al. U.S. Pat. No. 4,596,275. The sensitivity of migration type assays is frequently reduced, however, by the presence or formation in the sample of undesirable solid components which block the passage of labeled analyte to the detection zone. Assay sensitivity also declines when migration assay devices are flooded with too much liquid sample.

Migration assay devices usually incorporate within them reagents which have been attached to colored labels (i.e., conjugates), thereby permitting visible detection of the assay results without addition of further substances. See, for example, Bernstein, U.S. Pat. No. 4,770,853. Among such labels are gold sol particles such as those described by Leuvering in U.S. Pat. No. 4,313,734, dye sol particles such as described in U.S. Pat. No. 4,373,932 by Gribnau et al., dyed latex such as described by May et al., WO 88/08534, and dyes encapsulated in liposomes by Campbell et al., U.S. Pat. No. 4,703,017. These colored labels are generally limited in terms of the immobilization methods which are suitable. Moreover, they require a relatively large amount of ligand molecule and can involve expensive reagents, thereby adding to the cost.

More recently, “fourth generation” rapid detection immunoassay devices have been introduced. The “fourth generation” devices are intended to detect both antigens and antibodies for particular diseases. However, the “fourth generation” devices suffer from the same problems of the previously described devices.

### SUMMARY OF THE INVENTION

The invention provides a fourth generation rapid detection immunoassay device where the analytes migrate along different paths than conjugate-carrying buffer solutions. The immunoassay device of the invention is highly sensitive and provides accurate results while using small sample volumes. The device of the invention may be used with different types of body fluids and is useful in conjunction with the detection of many different diseases.

In accord with these objects, which will be discussed in detail below, both dry and liquid conjugate immunoassay device systems are provided. The systems of the invention include test cells having a first buffer-receiving location which receives a buffer solution and a first sorbent material defining a first horizontal flow path for the first buffer solution, a second sorbent material defining a second horizontal flow path distinct from said first horizontal flow path for the same or a different buffer solution provided to the first buffer-receiving location or to a second buffer-receiving location, a third sorbent material defining a third horizontal flow path for a sample provided at a sample-receiving location, said third horizontal flow path being distinct from said first and second horizontal flow paths, a fourth flow path for the sample provided at the sample-receiving location, said fourth horizontal flow path being distinct from said first, second, and third horizontal flow paths, a first test line or test site with one of immobilized antigens or antibodies located in a first test zone at a junction of the first and third sorbent materials, and a second test line or test site with the other of the immobilized antigens or antibodies located in a second test zone at a junction of the second and fourth sorbent materials. For purposes herein, the term “distinct” when used in conjunction with the words “flow path” or “migration path” shall be understood to mean “not in fluid communication except either (i) via a test zone, or (ii) at a buffer receiving or sample receiving location”.

Where the test cell of the invention is provided in a housing, the housing is provided with a first opening adjacent the first buffer-receiving location and a sample-receiving opening adjacent the sample receiving location. Where a second buffer-receiving location is utilized, a second buffer-receiving opening is provided in the housing adjacent the second buffer-receiving location. A first viewing window is provided in the housing above the first test line and a second viewing window is provided in the housing above the second test line.

In the preferred embodiment of the invention, the third sorbent material and fourth sorbent material are separate pieces which are coupled to a single sample receiving pad. Alternatively, if desired, the third and fourth sorbent materials can be integral with each other. Also, in the preferred embodiment, the first sorbent material and second sorbent material are separate pieces which may be coupled to the same buffer receiving pads or to two different buffer receiving pads. However, if desired, in an embodiment where a single buffer receiving pad is utilized, the first and second sorbent materials can be integral with each other. In the preferred embodiment, a control line or site is provided adjacent each test site.

In one embodiment of the invention, the materials, thicknesses and lengths of the first, second, third and fourth sorbent materials are chosen to adjust the timing regarding the liquid sample and liquid buffer reaching the test site.

In the dry conjugate system of the invention, a first dry conjugate is provided between the first opening and the test site. The first dry conjugate is supported on or within the first sorbent material such that when a buffer is added in the first opening, the first sorbent material wicks the buffer to the first conjugate which is then carried by the buffer to the first test site. A second dry conjugate is likewise supported on or within the second sorbent material such that when buffer is added in the first or second opening (if provided), the second sorbent material wicks the buffer to the second conjugate which is then carried by the buffer to the second test site. In the liquid conjugate system of the invention, a first buffer-conjugate liquid subsystem is provided and applied to the first opening. The first sorbent material then wicks the first buffer-conjugate subsystem to the first test site. A second buffer-conjugate liquid subsystem is provided and applied to the second opening. The second sorbent material then wicks the second buffer-conjugate subsystem to the second test site.

It will be appreciated that the system of the invention can be used in conjunction with different types of samples such as blood, urine, saliva, and feces, and can be used to test for the presence of any ligand. Where blood, saliva or feces is to be provided, the blood, saliva or feces may be diluted or mixed with buffer prior to being added through the sample-receiving hole. Alternatively, in some cases, the sample may be added through the sample-receiving hole and then a diluent may be added through the same hole.

The test cell of the invention is advantageous over the prior art because the test cell of the invention overcomes aggregation/agglutination problems between the conjugate(s) and the analyte in the sample which is a significant problem in traditional chromatographic immunoassay for relatively large analytes such as bacteria or aggregated viruses. In particular, in traditional chromatographic immunoassays, the complex between bacteria and conjugated antibody has difficulty migrating to the test line(s) and tends to remain in the bottom of test strip or in the pad. In this invention there is no complex binding between analyte and the conjugate until the sample reaches the test site(s), as the analyte is applied via its own paths to the test sites while the conjugates migrate by themselves. As a result, the system of the invention is extremely sensitive and specific.

Additional objects and advantages of the invention will become apparent to those skilled in the art upon reference to the detailed description taken in conjunction with the provided figures.

### BRIEF DESCRIPTION OF THE DRAWINGS

FIG. 1 is a top view of a first embodiment of the invention.

FIG. 1A is a schematic view of the antibody test portion of the first embodiment of FIG. 1.

FIG. 1B is a schematic view of the antigen test portion of the first embodiment of FIG. 1.

FIG. 2 is a top view of the first embodiment of the invention contained within a housing.

FIG. 3 is a schematic top view of a second embodiment of the invention.

FIG. 4 is a top view of the second embodiment of the invention contained within a housing.

FIG. 5 is a top view of the second embodiment of the invention adapted to test for dengue fever antibodies and antigens and contained within a housing.

FIG. 6 is a top view of the second embodiment of the invention adapted to test for HIV and Tuberculosis antibodies and antigens and contained within a housing.

FIG. 7 is a top view of the second embodiment of the invention adapted to test for multiple antibodies and antigens and contained within a housing.

### DETAILED DESCRIPTION OF THE PREFERRED EMBODIMENTS

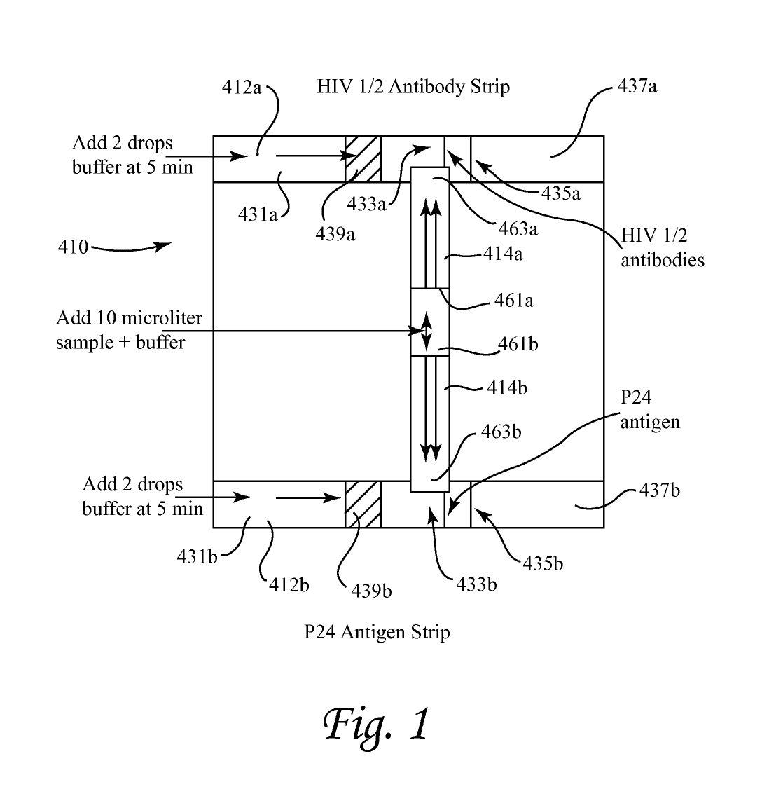

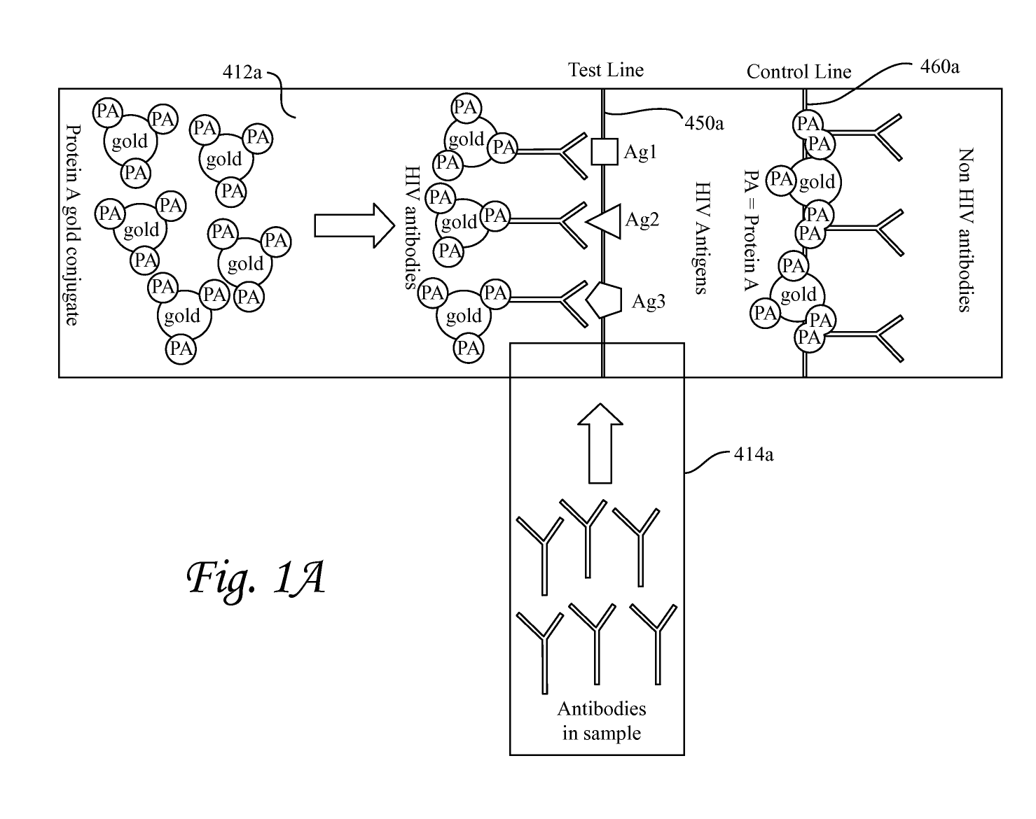

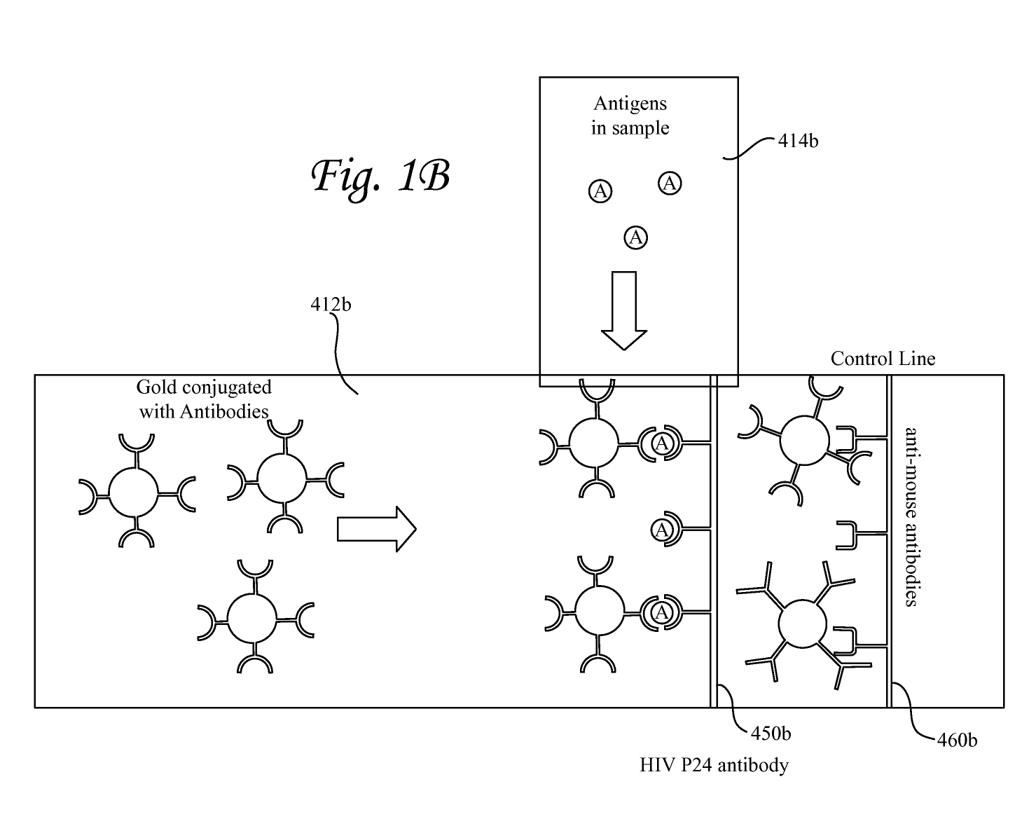

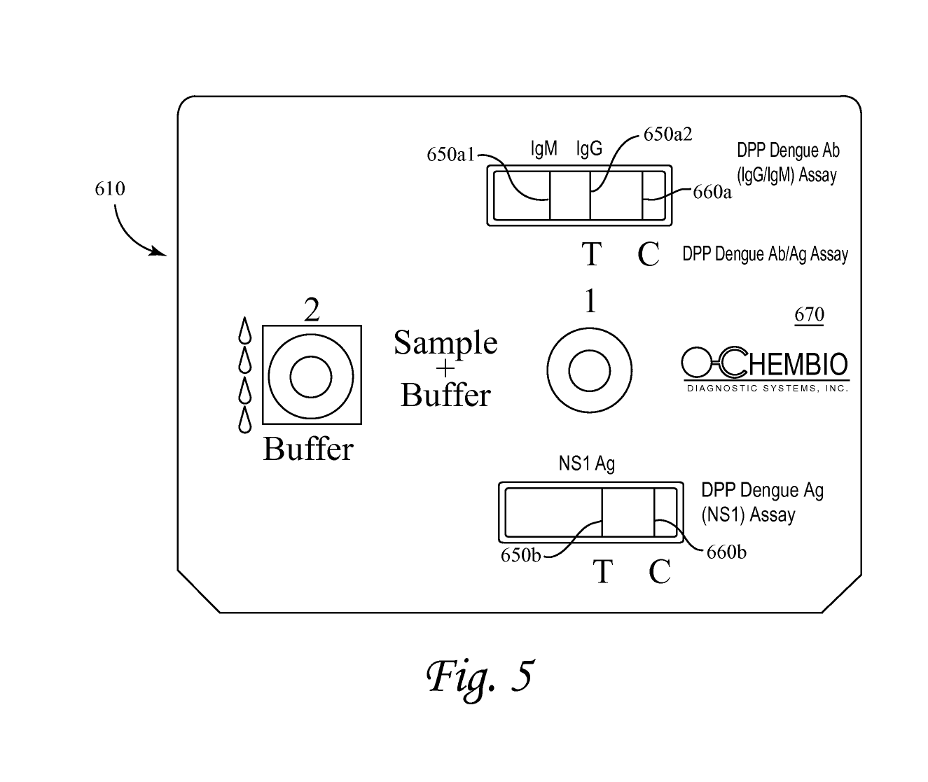

Turning now to FIGS. 1, 1A and 1B, a fourth generation immunoassay test device 410 is provided. Test device 410 is shown as a test device for testing HIV1/2 antibodies and P24 antigens (i.e., it is an HIV detection device), although as discussed with respect to FIGS. 5-7 below, many other types of antibody/antigen tests may be provided using the invention. Test device 410, as shown, includes first, second, third, and fourth sorbent or bibulous materials 412a, 412b, 414a, 414b defining first, second, third and fourth horizontal flow paths. The first sorbent material 412a preferably includes least two and preferably three or four zones and may be made from a plurality of materials. A first zone 431a (sometimes called a filter zone) is located at a first end of the strip 412a and extends to a second zone 433a (sometimes called a test zone) which is located at the junction with the second sorbent material 414a. The first zone 431a may constitute or have a filter (not shown) attached, and may have a conjugate 439a having desired antigens (e.g., protein A) with attached colored markers deposited and immobilized thereon or on a conjugate pad (not shown) attached thereto. The first sorbent material may constitute a thin membrane of sorbent or bibulous material typically made from nitrocellulose with a plastic backing (not shown). The first zone 431a is adapted to receive a buffer solution, to cause the buffer solution to contact the conjugate, thereby mobilizing the conjugate, and to wick the conjugate-carrying buffer solution to the second zone 433a. The second (test) zone 433a includes a second portion of the thin membrane which is preferably printed with a test line 450a (FIG. 1A) having immobilized antigens on the membrane as is well known in the art. An optional third zone 435a (sometimes called a control zone) which includes a third portion of the thin membrane may also be printed with a control line 460a (FIG. 1A) typically containing antibodies to the conjugate antigens as is well known in the art. If desired, an optional fourth zone 437a (sometimes called a reservoir zone) may be provided as a wicking reservoir as is also well known in the art. The fourth zone 437a may include a relatively thicker absorbent paper (not shown). Preferably underlying and/or overlying all the zones is a thin, preferably transparent plastic film or card (not shown) having an adhesive which keeps the sorbent materials in place. The card may be cut with an opening at the end of strip 412a so that it does not block liquid access to first sorbent strip 412a.

The second sorbent material 412b likewise preferably includes at least two and preferably three or four zones and likewise may be made from a plurality of materials. A first zone 431b (sometimes called a filter zone) is located at a first end of the strip 412b and extends to a second zone 433b (sometimes called a test zone) which is located at the junction with the second sorbent material 414b. The first zone 431b may constitute or have a filter (not shown) attached, and may have a conjugate 439b having desired antibodies with attached colored markers deposited and immobilized thereon or on a conjugate pad (not shown) attached thereto. The first sorbent material may constitute a thin membrane of sorbent or bibulous material typically made from nitrocellulose with a plastic backing (not shown). The first zone 431b is adapted to receive a buffer solution, to cause the buffer solution to contact the conjugate, thereby mobilizing the conjugate, and to wick the conjugate-carrying buffer solution to the second zone 433b. The second (test) zone 433b includes a second portion of the thin membrane which is preferably printed with a test line 450b (FIG. 1B) having immobilized P24 antibodies on the membrane as is well known in the art. An optional third zone 435b (sometimes called a control zone) which includes a third portion of the thin membrane may also be printed with a control line 460b (FIG. 1B) typically containing anti-mouse antibodies (if the conjugate 439 uses mouse antibodies) as is well known in the art. If desired, an optional fourth zone 437b (sometimes called a reservoir zone) may be provided as a wicking reservoir as is also well known in the art. The fourth zone 437b may include a relatively thicker absorbent paper (not shown). Preferably underlying and/or overlying all the zones is a thin, preferably transparent plastic film or card (not shown) having an adhesive which keeps the sorbent materials in place. The card may be cut with an opening at the end of strip 412b so that it does not block liquid access to first sorbent strip 412b.

The third sorbent material 414a may also be made from a plurality of materials and preferably includes two zones 461a, 463a. The first zone 461a (sometimes called a filter zone) may include a filter or pad (not shown) and a first portion of a thin membrane or sorbent or bibulous material typically made from nitrocellulose with a backing (not shown). The first zone 461a is intended to receive the sample at its first end and the first zone extends to the second zone 463a. The second zone 463a includes a second portion of the thin membrane which is in contact with the second zone 433a of the first sorbent material 412a. As suggested in FIGS. 1 and 1A, the first sorbent material 412a and the third sorbent material 414a are arranged such that the membranes are in contact with each other (they form a juncture) such that the test line 450a is effectively located between the membranes (as opposed to the backings contacting the membranes or each other). Thus, test line 450a could be printed on the second zone 463a of the third sorbent material 414a instead of, or in addition to the second zone 433a of the first sorbent material 412a. If desired, a thin plastic film or card (not shown) having an adhesive which keeps the third sorbent material in place may be utilized.

The fourth sorbent material 414b may also be made from a plurality of materials and preferably includes two zones 461b, 463b. The first zone 461b (sometimes called a filter zone) may include a filter or pad (not shown) and a first portion of a thin membrane or sorbent or bibulous material typically made from nitrocellulose with a backing (not shown). The first zone 461b is intended to receive the sample at its first end and the first zone extends to the second zone 463b. The second zone 463b includes a second portion of the thin membrane which is in contact with the second zone 433b of the second sorbent material 412b. As suggested in FIGS. 1 and 1B, the second sorbent material 412b and the fourth sorbent material 414b are arranged such that the membranes are in contact with each other (as opposed to the backings contacting the membranes or each other), and such that the test line 450b is effectively located between the membranes. Thus, test line 450b could be printed on the second zone 463b of the fourth sorbent material 414b instead of, or in addition to the second zone 433b of the second sorbent material 412b. If desired, a thin plastic film or card (not shown) having an adhesive which keeps the third sorbent material in place may be utilized.

Where sorbent materials 412a, 412b, 414a, 414b are made from standard-type nitrocellulose membranes with a backing, it is desirable for the sample migration and buffer-conjugate migration membranes to have different pore sizes. For example, if strip 412a (for the conjugate migration) has a 3μ pore size, and membrane 414a (for the sample migration) has a 15μ pore size, sample applied to membrane 414a will tend to migrate and stay in the sample membrane 414a and will tend not to migrate into the conjugate membrane 412a. Furthermore, it may be desirable for membranes 412a and 412b to have different size pores. Thus, for example, the membrane carrying the antibody conjugate might be larger than the membrane carrying the antigen conjugate.

As seen in FIG. 1, the four sorbent materials assume an “H” shape with the first and second sorbent materials forming the sides of the “H”, and the third and fourth sorbent materials forming the middle cross-bar of the “H”. It will be appreciated that the four sorbent materials may be laid out in other arrangements.

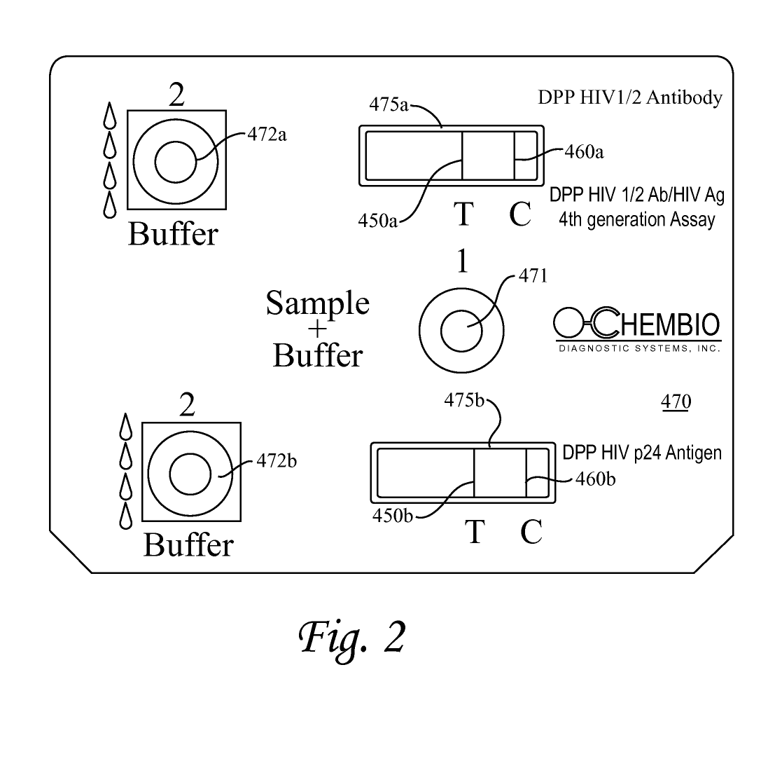

Turning to FIG. 2, it will be appreciated that the test device 410 of FIGS. 1, 1A and 1B can be provided inside a housing 470. Housing 470 is provided with a sample receiving hole 471 for receiving sample (and buffer if desired) and two buffer receiving holes 472a, 472b. Hole 471 is located directly above first zones 461a, 461b of sorbent strips 414a, 414b such that sample deposited in hole 471 will flow onto both sorbent strips 414a and 414b. Likewise, holes 472a and 472b are located directly above the first zones 431a, 431b of sorbent strips 412a, 412b such that buffer deposited in holes 472a, 472b will flow onto respective sorbent strips 412a, 412b. In addition, housing 470 is provided with windows 475a, 475b. Windows 475a, 475b are located above test lines 450a, 450b respectively so that the test lines may be seen through the windows. If desired, the windows may constitute clear plastic. Where third zones 435a, 435b are provided, windows 475a, 475b preferably extend above the respective control lines 460a, 460b.

It should be appreciated that because the same sample is to be provided to sorbent materials 414a, 414b, that the sorbent materials 414a, 414b optionally may be made from a single (integral) piece of material. If desired, a sample receiving pad (not shown) may be provided to receive the sample and provide the sample to the single piece of material or the individual pieces of material constituting the sorbent materials 414a and 414b.

The immunoassay 410 of FIG. 1 is preferably utilized as follows. First, a desired amount of sample (e.g., 10 microliters—not shown) possibly containing antibodies and/or antigens is provided to third and fourth sorbent materials 414a, 414b (through hole 471 if housing 470 is utilized) and allowed to migrate through the third and fourth sorbent materials 414a, 414b to their respective second zones 463a, 463b which are in contact with the second zones 433a, 433b of the first and second sorbent materials 412a, 412b respectively. Optionally, after providing the sample to the third and fourth sorbent materials, a preferably measured amount of liquid such as a buffer solution may be added (through hole 471 if housing 470 is utilized) to help in the migration of the sample. The amount of buffer added may depend upon the type of sample utilized (e.g., blood, urine, spit, etc.). Regardless, the sample reaches the test lines 450a, 450b which are printed atop the second zones 433a, 433b of the first and second sorbent material or infused therein. After a desired amount of time, by which time the HIV antibodies in the sample (if present) will have had an opportunity to bind to the HIV antigens immobilized at the test line 450a and HIV antigens in the sample (if present) will have had an opportunity to bind to the HIV P24 antibodies immobilized at the test line 450b, a preferably measured amount of liquid such as a buffer solution (not shown) is added to the first and second sorbent materials 412a, 412b (through holes 472a, 472b if housing 470 is utilized). It should be noted that different types of buffer solutions may be added through the respective holes, thereby permitting the sensitivity of the system to be optimized. After another period of time, sufficient to permit the conjugates 439a, 439b to migrate to the test sites 450a, 450b (and control sites 460a, 460b if provided), the test sites 450a, 450b (and control sites 460a, 460b if provided) are inspected (via windows 475a, 475b if housing 470 is utilized) in order to determine whether the sample is “positive” or not. Typically, a “positive” test indicating the presence of the HIV1 or HIV2 antibody in the sample is obtained when both the test site 450a and the control site 460a show lines of color. A “negative” test indicating the lack of the presence of the antibody in the sample is obtained when only the control site 460a shows a line of color. Similarly, a “positive” test indicating the presence of the HIV p24 antigen in the sample is obtained with both test site 450b and control site 460b show lines of color, while a “negative” test indicating the lack of the presence of the p24 antigen in the sample is obtained when only the control site 460b shows a line of color.

The method of the invention may be expedited by providing the housing 470 with numbering and/or lettering to indicate that hole 471 is for receiving the sample (and optionally some buffer) and is to be used first, and that holes 472a, 472b are for receiving the buffer solution and are to be used second.

Those skilled in the art will appreciate that the immunoassay 410 functions as follows. Because the test line 450a is provided with antigens immobilized on a membrane, if the test sample contains antibodies to the antigens, the antibodies will bind themselves to the antigens at the test line. Thereafter, when the conjugate 439a containing an antigen for the antibody coupled to a colored marker is caused to migrate to the test line, if the test sample contains the antibodies which are now held at the test line 450a, the antigen of the conjugate will bind itself to the antibodies and the colored marker will cause a colored line to appear at the test site 450a. If the test sample does not contain antibodies, the conjugate will not have the antibodies to bind to at the test line 450a, and no colored line will appear at the test site 450a. On the other hand, because the control line 460a is provided with antibodies, the antigens of the conjugate will always bind to the antibodies in the control line 460a, thereby causing a colored line to appear at the control site 460a if the conjugate reaches the control site 460a. Thus, if sufficient buffer solution is provided to the test cell, a colored line should always appear at the control site 460a, thereby providing a control for the test. Similarly, because the test line 450b is provided with antibodies immobilized on a membrane, if the test sample contains antigens to the antibodies, the antigens will bind themselves to the antibodies at the test line 450b. Thereafter, when the conjugate 439b containing an antibody for the antigen coupled to a colored marker is caused to migrate to the test line, if the test sample contains the antigens which are now held at the test line 450b, the antibody of the conjugate 439b will bind itself to the antigens and the colored marker will cause a colored line to appear at the test site 450b. If the test sample does not contain antigens, the conjugate will not have the antigens to bind to at the test line 450b, and no colored line will appear at the test site 450b. On the other hand, because the control line 460b is provided with selected antibodies (e.g., mouse antibodies) which are selected to be of the same type as the antibodies in the conjugate, the antibodies of the conjugate will always bind to the selected antibodies in the control line 460b, thereby causing a colored line to appear at the control site 460b if the conjugate reaches the control site 460b. Thus, if sufficient buffer solution is provided to the test cell, a colored line should always appear at the control site 460b, thereby providing a control for the test.

According to other embodiments of the invention, instead of providing dry conjugate deposits 439a, 439b having desired antigens or antibodies with attached colored markers in the test cell (e.g., on the sorbent materials 412a, 412b), the test cell does not include a dry conjugate at all. Rather, (wet) buffer-conjugate subsystems are utilized. Thus, after the sample has been deposited on sorbent materials 414a, 414b, a first buffer-conjugate subsystem (utilizing a conjugate with an antigen plus a buffer) is deposited on sorbent material 412a and permitted to migrate to the test line 450a, while a second buffer-conjugate subsystem (utilizing a conjugate with an antibody plus a buffer) is deposited on sorbent material 412b and permitted to migrate to the test line 450b.

According to further embodiments of the invention, instead of the viewing window being provided in the top of the housing, a window is provided in the bottom of the housing.

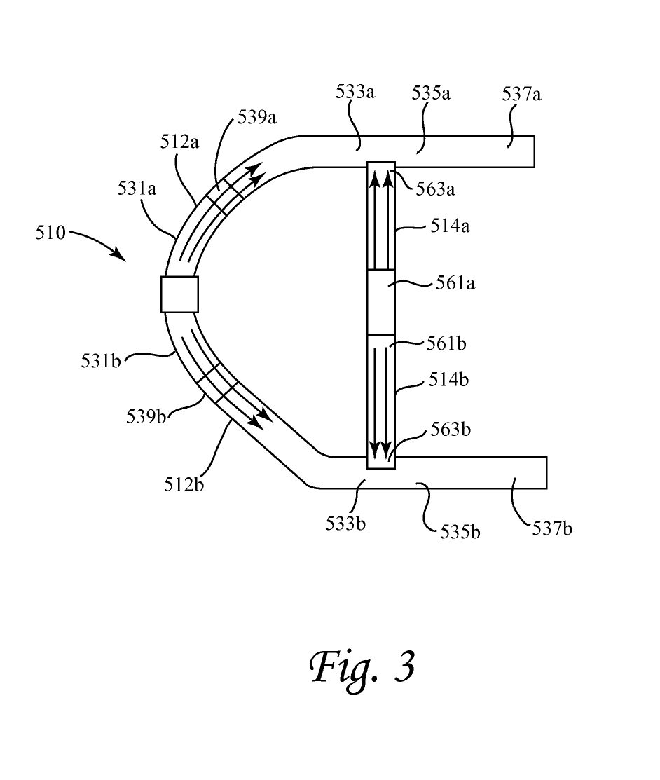

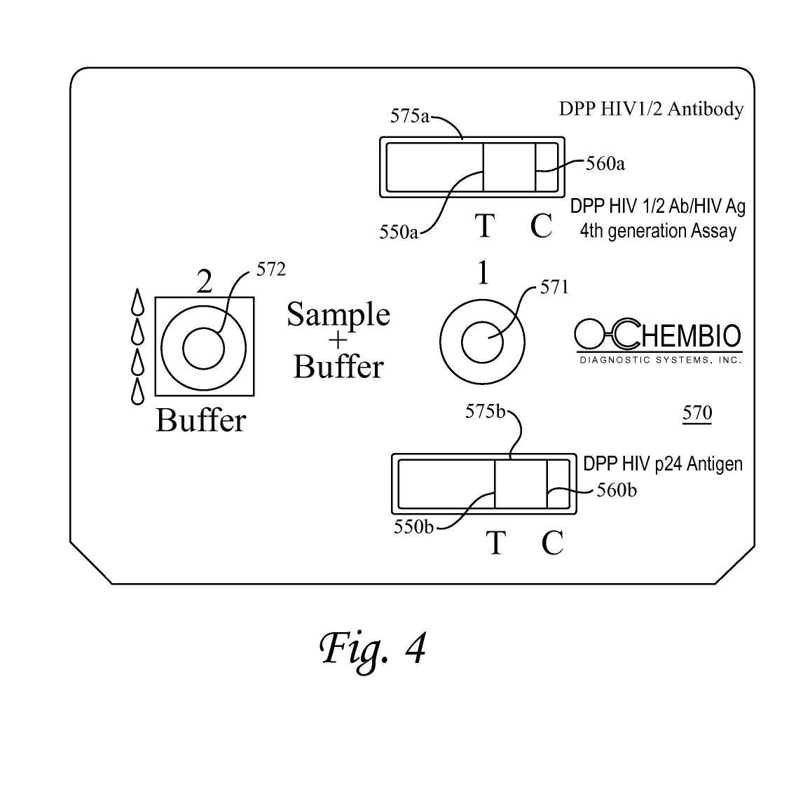

Turning now to FIG. 3, another embodiment of the invention is seen. Test device 510, as shown, includes first, second, third, and fourth sorbent or bibulous materials 512a, 512b, 514a, 514b defining first, second, third and fourth horizontal flow paths. The first sorbent material 512a preferably includes least two and preferably three or four zones and may be made from a plurality of materials. A first zone 531a (sometimes called a filter zone) is located at a first end of the strip 512a and extends to a second zone 533a (sometimes called a test zone) which is located at the junction with the second sorbent material 514a. The first zone 531a may constitute or have a filter (not shown) attached, and may have a conjugate 539a having desired antigens (e.g., protein A) with attached colored markers deposited and immobilized thereon or on a conjugate pad (not shown) attached thereto. The first sorbent material may constitute a thin membrane of sorbent or bibulous material typically made from nitrocellulose with a plastic backing (not shown). The first zone 531a is adapted to receive a buffer solution, to cause the buffer solution to contact the conjugate, thereby mobilizing the conjugate, and to wick the conjugate-carrying buffer solution to the second zone 533a. The second (test) zone 533a includes a second portion of the thin membrane which is preferably printed with a test line 550a (FIG. 4) having immobilized antigens on the membrane as is well known in the art. An optional third zone 535a (sometimes called a control zone) which includes a third portion of the thin membrane may also be printed with a control line 560a (FIG. 4) typically containing antibodies to the conjugate antigens as is well known in the art. If desired, an optional fourth zone 537a (sometimes called a reservoir zone) may be provided as a wicking reservoir as is also well known in the art. The fourth zone 537a may include a relatively thicker absorbent paper (not shown). Preferably underlying and/or overlying all the zones is a thin, preferably transparent plastic film or card (not shown) having an adhesive which keeps the sorbent materials in place. The card may be cut with an opening at the end of strip 512a so that it does not block liquid access to first sorbent strip 512a.

The second sorbent material 512b likewise preferably includes at least two and preferably three or four zones and likewise may be made from a plurality of materials. A first zone 531b (sometimes called a filter zone) is located at a first end of the strip 512b and extends to a second zone 533b (sometimes called a test zone) which is located at the junction with the second sorbent material 514b. The first zone 531b may constitute or have a filter (not shown) attached, and may have a conjugate 539b having desired antibodies with attached colored markers deposited and immobilized thereon or on a conjugate pad (not shown) attached thereto. The first sorbent material may constitute a thin membrane of sorbent or bibulous material typically made from nitrocellulose with a plastic backing (not shown). The first zone 531b is adapted to receive a buffer solution, to cause the buffer solution to contact the conjugate, thereby mobilizing the conjugate, and to wick the conjugate-carrying buffer solution to the second zone 533b. The second (test) zone 533b includes a second portion of the thin membrane which is preferably printed with a test line 550b (FIG. 4) having immobilized P24 antibodies on the membrane as is well known in the art. An optional third zone 535b (sometimes called a control zone) which includes a third portion of the thin membrane may also be printed with a control line 560b (FIG. 4) typically containing anti-mouse antibodies as is well known in the art. If desired, an optional fourth zone 537b (sometimes called a reservoir zone) may be provided as a wicking reservoir as is also well known in the art. The fourth zone 537b may include a relatively thicker absorbent paper (not shown). Preferably underlying and/or overlying all the zones is a thin, preferably transparent plastic film or card (not shown) having an adhesive which keeps the sorbent materials in place. The card may be cut with an opening at the end of strip 512b so that it does not block liquid access to first sorbent strip 512b.

It will be appreciated that first and second sorbent materials 512a, 512b are shaped with a curve or angle so that they can meet. As described hereinafter, this permits buffer (or a buffer plus conjugate subcomplex) to be added to a single location rather than two locations as described with reference to FIGS. 1 and 2. Thus, sorbent materials 514a, 514b are optionally made from a single (integral) piece of material.

The third sorbent material 514a may also be made from a plurality of materials and preferably includes two zones 561a, 563a. The first zone 561a (sometimes called a filter zone) may include a filter or pad (not shown) and a first portion of a thin membrane or sorbent or bibulous material typically made from nitrocellulose with a backing (not shown). The first zone 561a is intended to receive the sample at its first end and the first zone extends to the second zone 563a. The second zone 563a includes a second portion of the thin membrane which is in contact with the second zone 533a of the first sorbent material 512a. As suggested in FIG. 3, the first sorbent material 512a and the third sorbent material 514a are arranged such that the membranes are in contact with each other (as opposed to the backings contacting the membranes or each other), and such that the test line 550a is effectively located between the membranes. Thus, test line 550a could be printed on the second zone 563a of the third sorbent material 514a instead of, or in addition to the second zone 533a of the first sorbent material 512a. If desired, a thin plastic film or card (not shown) having an adhesive which keeps the third sorbent material in place may be utilized.

The fourth sorbent material 514b may also be made from a plurality of materials and preferably includes two zones 561b, 563b. The first zone 561b (sometimes called a filter zone) may include a filter or pad (not shown) and a first portion of a thin membrane or sorbent or bibulous material typically made from nitrocellulose with a backing (not shown). The first zone 561b is intended to receive the sample at its first end and the first zone extends to the second zone 563b. The second zone 563b includes a second portion of the thin membrane which is in contact with the second zone 533b of the second sorbent material 512b. As suggested in FIGS. 1 and 1B, the second sorbent material 512b and the fourth sorbent material 514b are arranged such that the membranes are in contact with each other (as opposed to the backings contacting the membranes or each other), and such that the test line 550b is effectively located between the membranes. Thus, test line 550b could be printed on the second zone 563b of the fourth sorbent material 514b instead of, or in addition to the second zone 533b of the second sorbent material 512b. If desired, a thin plastic film or card (not shown) having an adhesive which keeps the third sorbent material in place may be utilized.

Where sorbent materials 512a, 512b, 514a, 514b are made from standard-type nitrocellulose membranes with a backing, it is desirable for the sample migration and buffer-conjugate migration membranes to have different pore sizes. For example, if strip 512a (for the conjugate migration) has a 3μ pore size, and membrane 514a (for the sample migration) has a 15μ pore size, sample applied to membrane 514a will tend to migrate and stay in the sample membrane 514a and will tend not to migrate into the conjugate membrane 512a.

As seen in FIG. 3, the four sorbent materials assume an “A” shape with the first and second sorbent materials forming the sides of the “A”, and the third and fourth sorbent materials forming the middle cross-bar of the “A”. It will be appreciated that the four sorbent materials may be laid out in other arrangements.

Turning to FIG. 4, it will be appreciated that the test device 510 of FIG. 3 can be provided inside a housing 570. Housing 570 is provided with a sample receiving hole 571 for receiving sample (and buffer if desired) and a single buffer receiving holes 572. Hole 571 is located directly above first zones 561a, 561b of sorbent strips 514a, 514b such that sample deposited in hole 571 will flow onto both sorbent strips 514a and 514b. Likewise, hole 572 is located directly above the first zones 531a, 531b of sorbent strips 512a, 512b such that buffer deposited in hole 572 will flow onto respective sorbent strips 512a, 512b. In addition, housing 570 is provided with windows 575a, 575b. Windows 575a, 575b are located above test lines 550a, 550b respectively so that the test lines may be seen through the windows. If desired, the windows may constitute clear plastic. Where third zones 535a, 535b are provided, windows 575a, 575b preferably extend above the respective control lines 560a, 560b.

It should be appreciated that because the same sample is to be provided to sorbent materials 514a, 514b, that the sorbent materials 514a, 514b optionally may be made from a single (integral) piece of material. If desired, a sample receiving pad (not shown) may be provided to receive the sample and provide the sample to the single piece of material or the individual pieces of material constituting the sorbent materials 514a and 514b.

The immunoassay 510 of FIG. 3 is preferably utilized as follows. First, a desired amount of sample (e.g., 10 microliters—not shown) possibly containing antibodies and/or antigens is provided to third and fourth sorbent materials 514a, 514b (through hole 571 if housing 570 is utilized) and allowed to migrate through the third and fourth sorbent materials 514a, 514b to their respective second zones 563a, 563b which are in contact with the second zones 533a, 533b of the first and second sorbent materials 512a, 512b respectively. Optionally, after providing the sample to the third and fourth sorbent materials, a preferably measured amount of liquid such as a buffer solution may be added (through hole 571 if housing 570 is utilized) to help in the migration of the sample. The amount of buffer added may depend upon the type of sample utilized (e.g., blood, urine, spit, etc.). Regardless, the sample reaches the test lines 550a, 550b which are printed atop the second zones 533a, 533b of the first and second sorbent material or infused therein. After a desired amount of time, by which time the HIV antibodies in the sample (if present) will have had an opportunity to bind to the HIV antigens immobilized at the test line 550a and HIV antigens in the sample (if present) will have had an opportunity to bind to the HIV P24 antibodies immobilized at the test line 550b, a preferably measured amount of liquid such as a buffer solution (not shown) is added to the first and second sorbent materials 512a, 512b (through hole 572 if housing 570 is utilized). After another period of time, sufficient to permit the conjugates 539a, 539b to migrate to the test sites 550a, 550b (and control sites 560a, 560b if provided), the test sites 550a, 550b (and control sites 560a, 560b if provided) are inspected (via windows 575a, 575b if housing 570 is utilized) in order to determine whether the sample is “positive” or not. Typically, a “positive” test indicating the presence of the HIV1 or HIV2 antibody in the sample is obtained when both the test site 550a and the control site 560a show lines of color. A “negative” test indicating the lack of the presence of the antibody in the sample is obtained when only the control site 560a shows a line of color. Similarly, a “positive” test indicating the presence of the HIV p24 antigen in the sample is obtained with both test site 550b and control site 560b show lines of color, while a “negative” test indicating the lack of the presence of the p24 antigen in the sample is obtained when only the control site 560b shows a line of color.

The method of the invention may be expedited by providing the housing 570 with numbering and/or lettering to indicate that hole 571 is for receiving the sample (and optionally some buffer) and is to be used first, and that hole 572 is for receiving the buffer solution and is to be used second.

It will be appreciated by those skilled in the art that the embodiments of the invention may be realized using many different materials. For example, the sorbent material(s), which typically include a very thin, inert film, strip, sheet, or membrane may be formed from nitrocellulose, filter paper, silica, or from, e.g., microporous or microgranular woven or non-woven fabrics, or combinations thereof. Many types of suitable materials and combinations thereof are described in U.S. Pat. No. 4,960,691 to Gordon et al. and U.S. Pat. No. 4,956,275 to Zuk et al. which are both hereby incorporated by reference in their entireties. Often, the nitrocellulose or other sorbent materials will be provided with a thin non-porous inert plastic backing as previously described.

Thus, according to yet additional embodiments of the invention, the materials, thicknesses and lengths of the first, second, third and fourth sorbent materials are chosen to adjust the timing regarding the liquid sample and liquid buffer (or buffer-conjugate subsystem) reaching the test sites. By providing separate migration paths for the sample/analyte and the buffers or buffer-conjugate subsystems, the materials may also be chosen to enhance sensitivity of the system.

In a similar vein, it will be appreciated that the sorbent materials can be shaped in any of many manners and take any of many dimensions as is known in the art. Thus, in order to help expedite wicking, the sorbent material can be key-shaped with the strip having smaller width at the hole which receives the buffer solution and at the test site and control site, and a wider width at a reservoir zone. Such an arrangement is shown in U.S. Pat. No. 5,989,921 to Charlton et al., which is hereby incorporated by reference in its entirety herein. In any event, generally, the test strip will be substantially greater in length than in width, and substantially greater in width than in thickness. Indeed, in at least certain embodiments of the present invention, the strip at the test zone should be paper-thin (e.g., 0.1 mm thick) and sufficiently translucent such that the test and control lines can easily be seen through the test strip.

Further, the housing and the sorbent material can be integrated in an open lateral flow platform where injection molded polymer is provided with micro-pillars which enable exact control over flow by varying the height, diameter, shape and/or distance between the pillars. Such a platform essentially uses the same material for the housing and the sorbent wicking material and is sold by Amic AB of Uppsala, Sweden. See, e.g., www.amic.se. Since the injection molded polymer may be generally transparent, the entire housing may be considered the “window” through which the test and control lines/sites may be viewed.

It will also be appreciated that depending upon the type of test being constructed (e.g., pregnancy, HIV, tuberculosis (TB), prion, urine-analysis/drug, cardiac markers, cancer markers, Chagas, Chlamydia, dental bacteria (SM/LC), influenza A, influenza B, adenovirus, rotavirus, strep A, other bacteria or viruses, etc., and even veterinary applications such as CPV (canine parvovirus), FIV (feline immunodeficiency virus), FeLV (feline leukemia virus), and heartworm), the antibodies and antigens of interest will be different, and therefore the antigens and antibodies used in the test lines will need to be tailored accordingly. Likewise, the antigens or antibodies of the conjugates will need to be tailored accordingly. In some cases (such as HIV), the identical antigen may be utilized in the test strip as in the conjugate, as the binding site of the HIV antibody will bind with the HIV antigen at the test site and still provide additional binding sites for binding to the antigen-conjugate, while in other cases, different antigens might be required. Similarly, it will be appreciated that depending upon the type of test being constructed, the control site, where provided, will need to be tailored accordingly. Thus, for example, in an HIV antibody detection test, where the ligand being identified in the test zone will be the HIV 1 and/or HIV 2 antibodies, the antigen in the test zone can be a mixture of HIV 1 (e.g., gp41/gp120) and HIV 2 (gp36) peptides and/or recombinant antigens. The conjugate can be a colored latex or colloidal gold conjugated to protein A, Protein A/G, anti-human IgG/IgM, peptides or recombinant antigens.

Different types of tests are shown in FIGS. 5-7. FIG. 5 shows a fourth-generation assay for dengue (fever). The device 610 depicted in FIG. 5 is constructed in the same manner as the test device 510 of the second embodiment (FIG. 3) with a housing 670 similar to that used in the embodiment of FIG. 4, except that the for device 610, the test lines and conjugates utilized are different and the markings on the housing may be different. More particularly, device 610 utilizes a first set of test lines 650a1, 650a2 for detecting dengue antibodies (dengue Ab) in the sample. Test line 650a1 is provided with anti-human IgM antibody while test line 650a2 is provided with anti-human IgG antibody. As will be appreciated by those skilled in the art, IgM and IgG antibodies in the test sample will be captured at test lines 650a1 and 650a2 respectively regardless of whether the antibody is a dengue antibody or an antibody for other infectious diseases. However, the first conjugate provided in device 610 is a recombinant dengue antigen with a gold marker which will bind only to a dengue antibody. As a result, only if dengue IgM or dengue IgG antibodies are captured at test lines 650a1 and 650a2 will the conjugate be captured and show a positive test as seen in FIG. 5. Since the IgM antibody is an early marker which occurs during the early phase of infection, whereas the IgG typically first appears at a later stage of infection, the dengue test of FIG. 5 is provided with separate test lines 650a1 and 650a2 to detect dengue IgM and/or dengue IgG antibodies. However, if the distinction is not required, a single test line could be provided with a recombinant dengue antigen, and the conjugate could be any desired conjugate which will bind to the dengue IgM and dengue IgG antibodies which are captured at the test lines. Preferably, a first control line 660a is provided downstream of the test lines 650a1, 650a2.

As seen in FIG. 5, test device 610 also includes a dengue antigen (dengue Ag) detection test line 650b. Test line 650b is provided with anti-dengue NS1 (non-structural glycoprotein) monoclonal antibody which will capture the NS1 antigen. The second conjugate for the device 610 is preferably the same or a different monoclonal antibody against NS1 antigen which is conjugated to a marker such as gold sol. A second control line 660b is preferably provided as well.

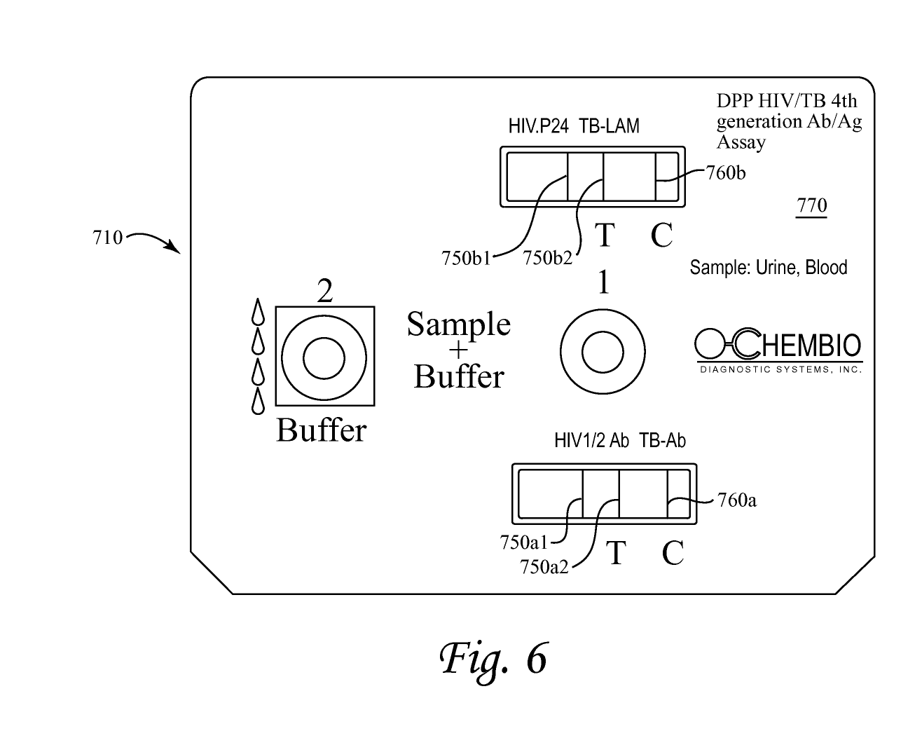

FIG. 6 shows a fourth-generation assay for HIV and tuberculosis (TB). The device 710 depicted in FIG. 6 is constructed in the same manner as the test device 510 of the second embodiment (FIG. 3) with a housing 770 similar to that used in the embodiment of FIG. 4, except that the for device 710, the test lines and conjugates utilized are different, the markings on the housing may be different, and the antigen detection is conducted in conjunction with the first and third sorbent strips while the antibody detection is conducted in conjunction with the second and fourth sorbent strips. More particularly, device 710 utilizes a first set of test lines 750a1, 750a2 for detecting HIV1/2 and TB antibodies in the sample. Test line 750a1 is provided with HIV1 and HIV2 recombinant antigen or synthetic peptides, while test line 750a2 is provided with a recombinant TB antigen cocktail or fusion. The first conjugate for the antibody detection test is preferably a protein A and/or anti-human IgM conjugate with gold sol. As a result, if HIV1 or HIV2 antibodies are in the sample, the antibodies will be captured by the antigen or synthetic peptides at test line 750a1, and if there are TB antibodies in the sample, they will be captured by the antigen cocktail or fusion at test line 750a2. When buffer is added to the test device 710 and the protein A and/or anti-human IgM conjugate migrates to test lines 750a1, 750a2, the conjugate will be captured by the HIV1 or HIV2 antibodies if they are present at test line 750a1 and by the TB antibodies if they are present at lest line 750a2. Preferably, a first control line 760a is provided downstream of the test lines 750a1, 750a2.

Test device 710 also includes test lines 750b1, 750b2 for testing for HIV.P24 antigens and TB-LAM (lipoarabinomannan) antigens respectively. More particularly, test line 750b1 is provided with an anti-HIV.P24 monoclonal and/or polyclonal antibody which will capture the p24 HIV antigen, while antigen test line 750b2 is provided with a LAM antibody which will capture a TB LAM antigen. The “second conjugate” used for the antigen detection in the test device 710 is a mixture of conjugates including a conjugate of an anti-HIV.P24 monoclonal or polyclonal antibody conjugated with a marker such as gold sol, and a conjugate of a LAM antibody conjugated with the same or a different marker used in the anti-HIV.P24 monoclonal or polyclonal conjugate. If a different marker is used for the LAM antibody conjugate, the color appearing at test line 750b2 can be different than the color appearing at test line 750b1 when both HIV and TB antigens are present. A second control line 660b is preferably provided as well.

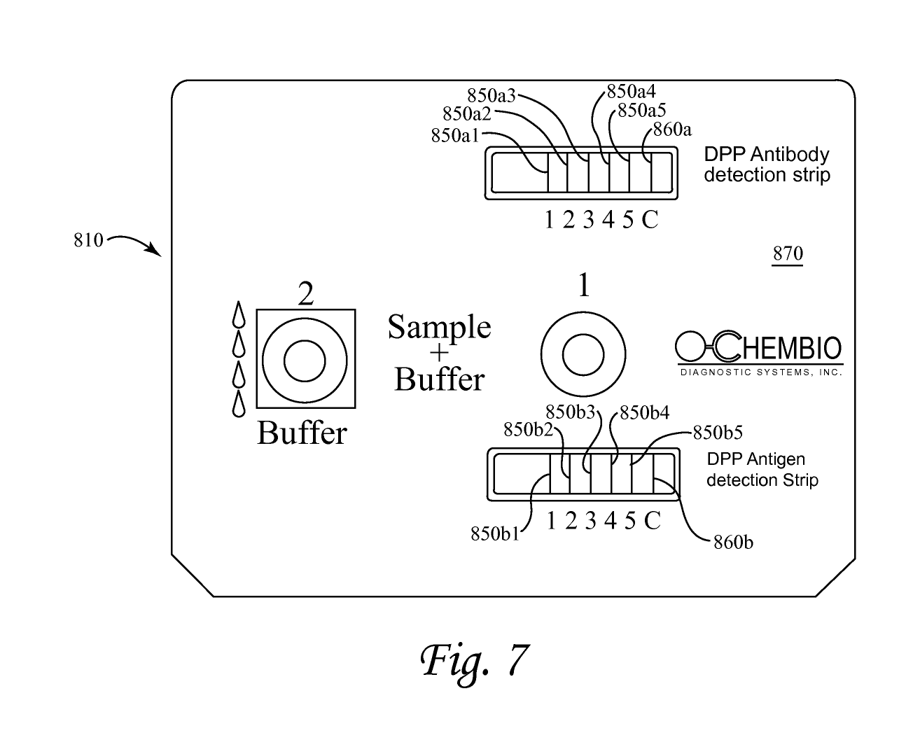

A fourth-generation assay for multiple antibody and multiple antigen tests is shown generically in FIG. 7. Test device 810 is constructed in the same manner as the test device 510 of the second embodiment (FIG. 3) with a housing 870 similar to that used in the embodiment of FIG. 4, except that the for device 810, the test lines and conjugates utilized are different and the markings on the housing may be different. As seen in FIG. 7, five test lines 850a1, 850a2, 850a3, 850a4, 850a5 are provided with different antigens in order to detect different antibodies, while five additional test lines 850b1, 850b2, 850b3, 850b4, 850b5 are provided with different antibodies to detect different antigens. The test device 810 could be used as a test for detecting allergies or multiple infectious diseases.

With respect to all of the embodiments of the invention, it will be appreciated by those skilled in the art that the marker of the conjugate may take many forms including different types of metal sols, a colored latex, any of various enzymes, etc. While the preferred embodiment of the invention provides a detection signal readily visible to the unaided eye, it will be appreciated that the invention encompasses other markers which can be detectible by ultraviolet radiation or other techniques such a fluoroscopy. Thus, it will be appreciated that a system employing the test cells of the invention which are read by an automatic reader such as a fluoroscopic or digital reader can be provided.

The present invention provides improved sensitivity without compromising the specificity of the assay. The main reasons for the sensitivity improvement are an improved migration of the sample to the test zones due to the distinct migration paths, and the effective binding of the analyte to the binding sites in the test zones prior to the reaction of the conjugated markers with the test zone complexes. For example, in the case of an HIV test, HIV specific antibodies in the blood serum samples applied to the third sorbent strip will migrate to the first test zone and will bind to the HIV test line. No other immunoglobulin G (IgG) in the blood will bind to the HIV antigens immobilized in the test zone. When buffer solution is added to the first sorbent strip to cause the protein A conjugate with latex or gold to migrate to the test zone, the protein A conjugate will bind to the FC part of the HIV antibodies which are already captured by the HIV peptides at the test line. Because the binding between protein A and the FC part of the HIV antibodies is very strong, only a small amount of HIV antibody needs to be present in order to be detected. This is in contrast to the traditional lateral flow HIV test systems where all human IgG (including HIV antibodies) in the blood sample will bind to the protein A before migration to the test line, because protein A binds non-specifically all IgG. Thus, the entire protein A, IgG, gold/latex complex will migrate to the test line which contains the HIV antigens. Only the HIV antibodies, protein A, gold/latex conjugates will then bind to the HIV antigens. However, because of the large amount of non-related IgG in the samples and the small amount of HIV antibodies present, there is a risk that not enough HIV antibodies will bind to the protein A, and the colored line will not be visible. Similarly, where HIV specific antigens are present in the blood serum sample, the antigens migrating in the fourth sorbent strip will reach the second test zone and will bind to the second test line containing the HIV P24 antibody. When buffer solution is added to the second sorbent strip to cause the antibody/marker conjugate to migrate to the second test zone, the antibody in the conjugate will bind to the antigen already captured at the second test line.

There have been described and illustrated herein several embodiments of immunoassays and methods of their use. While particular embodiments of the invention have been described, it is not intended that the invention be limited thereto, as it is intended that the invention be as broad in scope as the art will allow and that the specification be read likewise. Thus, while the specification discusses ligand binding using antigen/antibody reactions, other ligand binding mechanisms such as aptamer binding, nucleic acid binding, enzymatic binding, etc. may also be used. Also, while the test cells are described as having a single line for testing for a single ligand, two lines for testing for two ligands, and five test lines for testing five ligands, it will be appreciated that different numbers of lines may be utilized for testing for different numbers of ligands. Further, while the test cells are described as having holes in the top wall of a housing for receiving the sample and the buffer-solution or buffer-conjugate subsystem, it will be appreciated that one or both holes may be provided in the end wall or side wall of the housing. Similarly, while the sorbent material was described as preferably including a thin plastic backing, it will be appreciated that the plastic backing could be provided only at certain locations or not be provided at all. Where only partial backings or no backings are provided, the test and control sites can be located on either or both sides of the sorbent material. Further yet, while the test area and control are are shown as including test and control lines, it will be appreciated that the test and control sites can be configured differently such as in circles, squares, ovals, a broken line, etc. In fact, the test site and control site can be configured differently from each other. Also, while the invention was described as utilizing sorbent materials which are perpendicular to each other, it will be appreciated that the sorbent materials need not be perpendicular to each other, provided that distinct migration paths are provided for the analyte/sample and the buffer-conjugate subsystems. Those skilled in the art will also appreciate that the housing may be modified in additional ways to include separate windows for each test line. Also, while the invention was described in conjunction with the use of a buffer solution which is added to the migration path of the conjugate and optionally to the migration path of the sample, it will be appreciated that that one or more buffers may be chosen as desired to be added to the migration paths depending upon the test or tests to be conducted. Thus, buffers such as phosphate buffers or TRIS (tris hydroxymethylaminomethane) buffers are often utilized. However, the invention is intended to encompass the use of any diluent including water. In addition, the diluent may, if needed, may be added to and mixed with the sample prior to adding the sample to the sorbent material or the sample may be deposited first and the diluent may be added thereafter. Likewise, any diluent capable of causing conjugate to migrate may be utilized, and may be premixed with the conjugate in a liquid conjugate system, or provided to the migration path for the conjugate in a dry conjugate system. Further, it will be appreciated that the aspects of the assays and methods disclosed herein may be utilized in conjunction with any of the aspects and teachings contained in previously incorporated U.S. Ser. No. 11/908,071. It will therefore be appreciated by those skilled in the art that yet other modifications could be made to the provided invention without deviating from its spirit and scope as claimed.

What is claimed is:

1. An immunoassay device for testing a sample, comprising:
first, second, third, and fourth sorbent or bibulous materials defining first, second, third and fourth horizontal flow paths, the first and second flow paths providing for migration of first and second conjugates, and the third and fourth flow paths for the migration of the sample, wherein a first test area for detecting the presence of one or more different antibodies in the sample is located at the juncture of the first and third flow paths, and a second test area for detecting the presence of one or more different antigens in the sample is located at the juncture of the second and fourth flow paths.

2. An immunoassay device according to claim 1, further comprising:
a housing containing said first, second, third and fourth sorbent or bibulous materials, said housing having a first hole for receiving the sample, and at least one second hole for receiving liquid which causes migration of said first and second conjugates.

3. An immunoassay device according to claim 2, wherein:
said at least one second hole is a single second hole.

4. An immunoassay device according to claim 1, wherein:
said first and second sorbent or bibulous materials are integral with each other.

5. An immunoassay device according to claim 1, wherein:
said third and fourth sorbent or bibulous materials are integral with each other.

6. An immunoassay device according to claim 1, wherein:
said first, second, third and fourth sorbent or bibulous materials are laid out in one of an “H” shape and an “A” shape.

7. An immunoassay device according to claim 1, wherein:
said first test area includes a plurality of detection lines for detecting the presence of a plurality of different antibodies.

8. An immunoassay device according to claim 7, wherein:
said second test area includes a plurality of detection lines for detecting the presence of a plurality of different antigens.

9. An immunoassay device according to claim 1, wherein:
said first sorbent or bibulous material has a first pore size, and said second sorbent or bibulous material has a second pore size, and said second pore size is larger than said first pore size.

10. An immunoassay device according to claim 9, wherein:
said third sorbent or bibulous material has a third pore size, and said third pore size is larger than said second pore size.

11. An immunoassay device according to claim 1, wherein:
said first test area includes a first test line containing HIV antigens, and said second test area includes a second test line containing HIV antibodies.

12. An immunoassay device according to claim 1, wherein:
said first test area includes a first test line containing IgM antibodies, and a second test line containing IgG antibodies, and said first conjugate comprises a dengue antigen with a marker.

13. An immunoassay device according to claim 12, wherein:
said second test area includes a third test line containing anti-dengue antibodies.

14. An immunoassay device according to claim 1, wherein:
said first test area includes a first test line containing HIV antigens or synthetic peptides, and a second test line containing TB antigens, and said second test area includes a third test line containing HIV antibodies and a fourth test line containing lipoarabinomannan (LAM) antibodies.

15. An immunoassay device according to claim 14, wherein:
said second conjugate comprises a mixture of an anti-HIV.P24 monoclonal or polyclonal antibody conjugated with a first marker, and a conjugate of a LAM antibody conjugated with a second marker.
